(19) United States
(12) Patent Application Publication
(10) Pub. No.: US 2003/0170706 A1
(43) Pub. Date: Sep. 11, 2003

(54) Use of a volatile hybridization wash buffer

(76) Inventors: Roland Green, Madison, WI (US); Michael Sussman, Madison, WI (US)

Correspondence Address: QUARLES & BRADY LLP, FIRSTAR PLAZA, ONE SOUTH PINCKNEY STREET, P.O BOX 2113 SUITE 600, MADISON, WI, 53701-2113, US

(21) Appl. No.: 10/355,782

(22) Filed: Jan. 31, 2003

Related U.S. Application Data
(60) Provisional application No. 60/353,841, filed on Feb. 1, 2002.

(51) Int. Cl. C12Q 1/68; C07H 21/02; C07H 21/04; G01N 1/30; G01N 33/48

(52) U.S. Cl. 435/6; 435/40.5; 536/23.1

(57) ABSTRACT

A method for washing and drying a surface that contains a salt and hybridized DNA or RNA molecules is disclosed. The method involves exposing the surface to a volatile buffer that does not denature a specific DNA/DNA, DNA/RNA or RNA/RNA hybridization on the surface, removing the volatile buffer, and evaporating residue volatile buffer.

### CROSS-REFERENCE TO RELATED APPLICATIONS

This application claims the benefit of U.S. Provisional Application No. 60/353,841 filed Feb. 1, 2002.

### STATEMENT REGARDING FEDERALLY SPONSORED RESEARCH OR DEVELOPMENT

Not applicable.

### BACKGROUND OF THE INVENTION

Hybridization between DNA and DNA, DNA and RNA, or RNA and RNA molecules has been used widely in many different applications such as disease gene detection. Recently, methods for making an array of a large number of DNA molecules in a very small area such as a microscope slide, the DNA microarray synthesis technology, has been developed. See, e.g., PCT patent publication Nos. WO 99/42813, 92/10092 and 90/15070, and U.S. Pat. No. 5,143,854, each of which is hereby incorporated by reference in its entirety. The DNA microarray synthesis technology makes it possible to conduct a very large number of hybridizations simultaneously. Thus, the DNA microarray technology can be used to simultaneously detect the existence of a large number disease genes and/or various forms of each disease gene. The DNA microarray technology can also be used to provide the complete nucleic acid sequence of a target DNA molecule. Applications of the DNA microarray synthesis technology usually involve synthesizing a DNA array, running a hybridization reaction between the DNA array and a DNA and/or RNA source, and scanning the DNA array to detect hybridization.

Most commercially available scanners that can be used or are already in use for scanning DNA or RNA microarrays require microarray samples to be dry microscope slides. These scanners include those manufactured by Axon, GeneFocus, GSI Iumonics, Genomic Solutions and Virtek. Two exceptions are the Affymetrix scanner and a scanner manufactured by Applied precision, which can collect data from microarray samples in solution. Dry scanning can be advantageous over wet scanning in that dry slides are easier to store and transport.

### BRIEF SUMMARY OF THE INVENTION

When dry scan is used to detect DNA/DNA, DNA/RNA or RNA/RNA hybridizations on a surface, the surface needs to be dried before it is subject to scanning. Salt from residue of the solutions that the surface has been exposed to may precipitate out and accumulate on the surface during the drying process and the accumulated salt can increase the background level of scan images of the surface. The present invention provides a method for washing a surface that contains a salt and hybridized DNA or RNA molecules to reduce the accumulation of salt precipitates on the surface during the drying process. The method involves exposing the surface to a volatile buffer that does not denature a specific hybridization on the surface, removing the volatile buffer, and evaporating residue volatile buffer. The volatile buffer has a pH value between about 6 and about 8 and contains a salt that evaporates during the drying process.

An object of the present invention is to reduce salt precipitates accumulated on a surface when the surface is dried without denaturing DNA/DNA, DNA/RNA or RNA/RNA hybridizations on the surface.

A feature of the present invention is to wash a surface with a volatile buffer.

An advantage of the present invention is that it does not denature even low stringency hybridizations such as those between short oligonucleotides.

Further objects, features and advantages of the invention will be apparent from the following detailed description when taken in conjunction with the accompanying claims.

### BRIEF DESCRIPTION OF THE SEVERAL VIEWS OF THE DRAWINGS

Not applicable.

### DETAILED DESCRIPTION OF THE INVENTION

Many applications of the DNA or RNA microarray technology involve hybridizing a DNA or RNA microarray with a DNA and/or RNA sample. When a dry scan of the microarray is used to detect hybridizations, drying the microarray prior to scanning is necessary. Depending on the mechanism of detection, several other steps between the hybridization reaction and the drying of the microarray may be necessary. For example, fluorescence is commonly used as a detection mechanism. In this case, the DNA or RNA sample that is hybridized against the microarray is first fluorescently labeled. By “fluorescently labeled,” we mean the DNA or RNA source is labeled with a compound that either fluoresce without further staining or fluorescence may be induced by a staining step. In the former situation, a washing step to remove the non-hybridized DNA or RNA molecules from the DNA or RNA source is necessary. In the latter situation, an additional staining step and an additional washing step for removing residue staining buffer components are necessary.

Regardless whether the detecting mechanism is fluorescence or what steps are necessary between the hybridization reaction and the drying of the microarray, the salts in a solution that contacts the DNA or RNA microarray right before the microarray is dried for scan can precipitate out during the drying process and accumulate on the surface where the microarray is formed. The accumulated salt precipitates can increase the background level of scan images of the microarray. Thus, it is preferable to remove salt before the microarray is dried.

Salt removal from a surface such as that of a microscope slide on which a microarray is formed is usually accomplished by washing the surface with a low salt washing buffer. When the microarray contains DNA or RNA molecules that have long hybridized sequences, low salt washes can be performed without disrupting hybridization. However, many microarrays such as oligonucleotide arrays contain DNA or RNA molecules with relatively short hybridized sequences. The hybridization between relatively short complementary sequences cannot withstand the stringent low salt wash. Other low stringency hybridizations, such as those containing mismatches, cannot withstand low salt washes either.

The present invention provides a method that removes salt but does not denature hybridized DNAs or RNAs, including short hybridized DNAs or RNAs. The buffer used in the method is a volatile buffer. By “volatile buffer,” we mean a buffer that contains a salt and which will evaporate during the drying process. Any salt that evaporates during the drying process can be used to make the volatile buffer. In one class of salts that can be used, the element(s) that replaces part or all of the hydrogens of an acid is an ammonia or an substituted ammonia such as amines and pyridines. An example of the salts of the present invention is ammonium acetate. Other examples of volatile buffers used in the present invention include but are not limited to collidine/acetic acetic acid, pyridine/glacial acetic acid (for example, 100 ml pyridine and 4 ml glacial acetic acid in 1 L water), triethanolamine/HCl, ammonia/formic acid, trimethylamine/CO₂, triethylamine/CO₂, ammonium carbonate/ammonia, NH₄HCO₃ (for example, 24 g NH₄HCO₃ in 1 L water).

The volatile buffer used in the present invention has a pH value between about 6 and about 8. Preferably, the pH value of the volatile buffer is between about 6.5 and about 7.5. One of ordinary skill in the art knows how to adjust the pH of a buffer.

The salt concentration of the volatile buffer should be high enough so that specific hybridizations will not be denatured. Since the salt in the volatile buffer can evaporate, even high salt concentrations will not leave salt deposit on a microarray during the drying process to increase the background level of scan images. By specific hybridization, we mean a hybridization that is desired to be detectable in a particular assay. For example, when one wants to detect all hybridizations with one or no mismatch in an assay, a hybridization with one mismatch is considered specific for this assay by the present invention. However, if one only wants to detect hybridizations with no mismatches in another assay, a hybridization with one mismatch is considered nonspecific by the present invention. The salt concentration of a volatile buffer used in the present invention only needs to be high enough so that a specific hybridization for a particular assay is not denatured. Of course, higher concentrations may also be used. When a specific hybridization in a particular assay includes hybridizations with lower stringency (shorter hybridized sequences and/or more mismatches), the minimum salt concentration required so that the volatile buffer will not denature a specific hybridization will be higher. The correlation between the salt concentration and the stringency of a washing buffer is within the knowledge of one of ordinary skill in the art. In addition, it is easy and one of ordinary skill in the art knows how to test whether a volatile buffer with a particular salt concentration will denature a particular hybridization. For example, one can easily test whether a volatile buffer will denature a 20 base pair hybridization by hybridizing two DNA sequences that are complementary to each other over a 20 base pair span and wash the hybridized DNA molecules with the volatile buffer. Generally speaking, when the salt concentration of a volatile buffer used in the present invention is above about 500 mM, washing microarrays with the volatile buffer will not denature a hybridization between two complementary sequences that are eighteen base pairs or longer in length at room temperature.

In a particular application, a volatile buffer of the present invention may be used to control hybridization stringency in addition to removing salts. To do this, the salt concentration of the volatile buffer used in the present invention should be in a range that allows nonspecific but not specific hybridized double stranded DNA or RNA molecules to denature. As mentioned above, it is within the knowledge of one of ordinary skill in the art as to the relationship between salt concentration and hybridization stringency so that one either knows the concentration range or it is easily determined.

A volatile buffer in the present invention may be used for the sole purpose of removing salts. The stringency of a hybridization is controlled by the hybridization condition, a conventional washing buffer such as MES, or otherwise. In this case, a volatile buffer of the present invention can have a salt concentration above about 500 mM so that it can be used to wash most microarrays without denaturing specific hybridizations.

A volatile buffer of the present invention may also be used to wash microarrays with high stringency hybridizations such as those between two DNA molecules with very long complementary sequences to remove salt thereof.

After washing a DNA or RNA microarray with a volatile buffer of the present invention, the volatile buffer is removed and the microarray is dried by evaporating residue volatile buffer. Any suitable drying method may be used. For example, the microarray can be left to air dry or a stream of gas such as argon gas can be applied to aid the drying process.

In the example shown below, 1 M ammonium acetate was used to wash a DNA microarray. Since 1 M ammonium acetate is low stringency, hybridized DNAs were not denatured by the wash. The wash removes salt left from earlier treatments that would have otherwise precipitated out to increase background level of scan images. Since ammonium acetate can evaporate, washing with it did not leave salt precipitates on the microarray.

In the case of ammonium acetate, the highest concentration that can be made is 7.5 M. The preferred concentration range for washing is between 100 mM and 7.5 M. When the concentration is below 100 mM, hybridizations of 18 base pairs or shorter may be denatured. The most preferred concentration range for washing is between 500 mM and 1 M. In this range, the concentration is high enough so that almost no hybridization will be denatured. Regardless of water ammonium acetate concentrations, the pH value should be around 7.5. In practice, due to variations in handling, the pH value may vary between 6.8 and 7.7, which works fine for most washes.

The washing method of the present invention for reducing salt level is not limited to DNA or RNA microarrays. For any surface that contains hybridized DNA or RNA molecules, if the hybridization detection method requires the surface to be dried and salt precipitation and accumulation occur on the surface during the drying process to increase background level of detection, the washing method of the present invention can be useful. An example other than a DNA or RNA microarray that the washing method of the present invention can be applied to is in situ hybridization of a fluorescently labeled DNA or RNA probe to DNAs or RNAs in a slice of tissue sample from an animal fixed on a microscope slide. Another example is in situ hybridization of a fluorescently labeled DNA or RNA probe to a chromosome preparation from an animal fixed on a microscope slide. Washing the slide with a volatile buffer of the present invention will help to reduce salt precipitates accumulation on the slide during the drying process which can increase the background level of scan images of the slide.

### EXAMPLE

Materials and Methods

1. Materials: Acetylated Bovine Serum Albumin (BSA) solution [50 mg/mL] was obtained from Gibco BRL Life Technologies, cat# 15561-020. Herring Sperm DNA was obtained from Promega/Fisher Scientific, cat# D1811. Micropure Separator was obtained from Amicon, cat# 42512. Control Oligo B2 (control Oligo for the antisense probe array and HPLC-purified) has the following sequence: 5′-bio GTCGTCAAGATGCTACCGTTCAGGA-3′. 5M NaCl Rnase-free and Dnase-free was obtained from Ambion, cat# 9760. MES Free Acid Monohydrate SigmaUltra was obtained from Sigma Chemical, cat# M5287. MES Sodium Salt was obtained from Sigma Chemical, cat# M3885. EDTA Disodium Salt 0.5 M solution (100 mL) was obtained from Sigma Chemical, cat# E7889. Molecular Biology Grade Water was obtained from Biowhittaker, cat# 16-001Y. Tween-20 10% was obtained from Pierce Chemical, cat# 28320.

2. Solution preparation: 12×MES Stock was prepared by mixing the following and adjust the final volume to 100 mL with water (1.22M MES, 0.89M [Na+]): 70.4 g MES free acid monohydrate, 193.3 g MES Sodium Salt and 800mL Molecular Biology Grade water. The pH should be between 6.5 and 6.7. The solution was filtered through a 0.2 um filter.

2×MES Hybridization Buffer—For 50mL (100 mM MES, 1M [Na+], 20 mM EDTA, 0.01% Tween-20), the following were mixed: 8.3 mL of 12×MES Stock, 17.7 mL of 5M NaCl, 4.0 mL of 0.5M EDTA, 0.1 mL of 10% Tween-20 and 19.9 mL of water.

Hybridization Cocktail—the following components were mixed for each hybridization:

| | Prehybridization | | | |
|---|---|---|---|---|
| Component | Solution | | Hybridization Solution | |
| Fragmented cRNA | — | | 16 | ug |
| Control Oligonucleotide B2 | — | | — | |
| (5 nM) | | | | |
| 1 uM CPK6 | — | | 0.4 | ul |
| Herring Sperm DNA | 4.0 | ul | 2.0 | ul |
| (10 mg/mL) | | | | |
| Acetylated BSA (50 mg/mL) | 4.0 | ul | 2.0 | ul |
| 2X MES Hybridization Buffer | 200 | ul | 100 | ul |
| Water | 192 | ul | to final volume of 200 | |
| | | | ul | |
| FINAL VOLUME | 400 | ul | 200 | ul |

When frozen stocks of the above cocktail was used, the stock was heated to 65° C. for 5 minutes to resuspend the cRNA completely before using.

The stain buffer was made as the following and protected from light: Before use, 1× stain buffer was prepared by mixing the following: 2× Stain buffer (500 ul), BSA (40 ul), SAC3 (10 ul) and water (450 ul).

3. The microaray was constructed of oligonucleotides 24 base pairs in length.

4. The microarray probes or oligos were based on genomic information from mouse and Drosophilia.

5. Prehybridization: The microarray was heated to 49° C. just before use. The prehybridization solution was heated to 99° C. for 6 minutes and then incubated at 49° C. (water bath) for 2 minutes. Next, the prehybridization solution was spun at room temperature for 5 minutes, maximum speed and was incubated at 49° C. (water bath) for 2 minutes. The prehybridization solution was applied to the prewarmed microarray and the microarray was incubated in the prehybridization solution in 49° C. hybridization oven for 20 minutes.

6. Hybridization: The hybridization solution was made and heated to 99° C. for 6 minutes. The hybridization solution was then incubated at 49° C. (water bath) for 2 minutes, spun at room temperature for 5 minutes, maximum speed, to spin down insoluble materials, and incubated at 49° C. (water bath) until the microarray was prehybridized. The prehybridization solution was removed from the microarray and 190 μl of hybridization cocktail was added to the microarray. Care should be taken to avoid adding any of the insoluble materials. The hybridization reaction was run for 16-20 hours, with rotation, in a 49° C. hybridization oven.

7. Staining and washing: The prehybridization solution was removed and NonStringent Buffer was immediately applied to the microarray. The microarray was incubated at room temperature for 10 minutes, during which the NonStringent Buffer was changed every 2 minutes. Then, 50° C. Stringent Buffer was applied to the microarray and the microarray was incubated on 50° C. heat block for 3 minutes, during which Stringent Buffer was changed every 45 seconds. The microarray was then washed with room temperature NonStringent Buffer. Next, the 1× Stain buffer was applied to the microarray and the staining reaction was run at room temperature (and in the dark) for 20 minutes, after which the stain solution was quickly tapped off. The microarray was inserted into a 50 mL Falcon tube containing 35° C. NonStringent buffer. The tube was dropped into 35° C. water bath for 5 minutes. The microarray was transferred to Falcon tube containing 1 M Ammonium Acetate. The tube was then capped and inverted several times. The microarray was transferred to a fresh tube with 1 M Ammonium Acetate and the tube was capped and inverted. The microarray was removed from Ammonium Acetate and quickly blown dry with high purity Argon. The microarray was scanned by an optical scanner.

Results

Experiments were performed to test the Ammonium Acetate wash. Arrays were made with sequences ranging from 18-24 bases. Mismatch controls wee included to determine specificity of hybridization. The NS wash removes the bulk of the non-specific target from the array. The stringent wash removes the close but not perfect match target from the array. If this wash is performed too long, perfect match sequence will also be removed.

To test the efficacy of the Ammonium Acetate, we hybridized arrays and then rinsed them under different conditions using NS wash, water, 1M ammonium acetate 0.5M ammonium acetate 0.1M ammonium acetate.

The 1M and 0.5M ammonium acetate slide had the highest signal to background. Signal was as high as with the NS wash, but the background was much lower. Also, the NS wash showed signs of physical damage as the result of salt crystals. The water wash showed low background and low signal. The 0.1M ammonium acetate showed low background and somewhat lower signal.

2× Stain Buffer (1×Concentration: 100 mM MES Salt and Free Acid Solution (See 12× MES Below), 1M [Na+], 0.05% Tween-20)

Recipe:

| | | |
|---|---|---|
| 41.7 | mL | 12X MES Stock Buffer (above) |
| 92.5 | mL | 5M NaCl |
| 2.5 | mL | 10% Tween-20 |
| 112.8 | mL | water |
| 250 | ml | |

Combine listed components and ring to volume in a graduated cylinder

Sterile filter, 0.2 μm.

NSWB Non-Stringent Wash Buffer (6× SSPE, 0.01% Tween-20)

Recipe:

| | | |
|---|---|---|
| 300 | mL | 20X SSPE |
| 1.0 | mL | 10% Tween-20 |
| 698 | mL | water |
| 1000 | ml | |

Combine listed components and bring to volume in a graduated cylinder

Sterile filter, 0.2 μm

SWB Stringent Wash Buffer (100 mM MES Salt and Free Acid Solution (See 12× MES Below), 0.1M [Na+], 0.01% Tween-20):

Recipe:

| | | |
|---|---|---|
| 83.3 | mL | 12X MES Stock Buffer (above) |
| 5.2 | mL | 5M NaCl |
| 1.0 | mL | 10% Tween-20 |
| 910.5 | mL | water |
| 1000 | ml | |

Combine listed components and bring to volume in a graduated cylinder

Sterile filter, 0.2 □m

12×MES Stock Buffer, 1L (1.22M MES, 0.89M [Na+])

Recipe:

| | | |
|---|---|---|
| 70.4 | g | MES, free acid monohydrate |
| 193.3 | g | MES, sodium salt |

molecular biology grade water to volume

| | |
|---|---|
| 1 | ml |

Combine MES salt and free acid, bring to volume with water

pH final should be 6.5-6.7

Sterile filter, 0.2 μm

We claim:

1. A method for washing a surface that contains a salt and hybridized DNA or RNA molecules, the method comprising the steps of:
exposing the surface to a volatile buffer that does not denature a specific hybridization on the surface;
removing the volatile buffer; and
evaporating residue volatile buffer.

2. The method of claim 1, wherein the surface is one on which a DNA or RNA microarray is formed.

3. The method of claim 1, wherein volatile buffer is selected from the group consisting of ammonium acetate, collidine-acetic acid solution, pyridine-glacial acetic acid solution, triethanolamine-hydrogen chloride solution, ammonia-formic acid solution, trimethylamine-carbon dioxide solution, triethylamine-carbon dioxide solution, NH₄HCO₃ solution and ammonium carbonate-ammonia solution.

4. The method of claim 1, wherein the volatile buffer is ammonium acetate.

5. The method of claim 4, where in the ammonium acetate has a concentration range of 0.1 molar to 7.5 molar.

6. The method of claim 4, where in the ammonium acetate has a concentration range of 0.5 molar to 2 molar.

7. The method of claim 1, wherein the evaporating of residue volatile buffer is aided by a stream of gas.

8. The method of claim 7, wherein the gas is argon gas.

9. The method of claim 1, wherein the surface is one on which a slice of animal tissue is fixed.

10. The method of claim 1, wherein the surface is one on which a chromosome preparation from an animal is fixed.

11. A method for detecting DNA/DNA, DNA/RNA or RNA/RNA hybridization on a DNA or RNA microarray, comprising the steps of:
providing a DNA or RNA microarray;
incubating the microarray with a source of fluorescently labeled DNA or RNA in a hybridization buffer;
removing the hybridization buffer;
exposing the microarray to a volatile buffer that does not denature a specific hybridization on the microarray;
removing the volatile buffer;
evaporating residue volatile buffer; and
scanning the microarray with a fluorescence scanner.

12. The method of claim 11, wherein the fluorescently labeled DNA or RNA fluoresces without further staining.

13. The method of claim 12, further comprising the step of:
washing the microarray with a washing buffer before exposing the microarray to the volatile buffer.

14. The method of claim 11, wherein the biotin labeled DNA or RNA needs staining to fluoresce.

15. The method of claim 14, further comprising the step of:
washing the microarray with a washing buffer before exposing the microarray to the volatile buffer.

16. The method of claim 15, further comprising the step of:
incubating the microarray with a staining buffer to bind fluorescent label to target oligos after washing the microarray with the washing buffer but before exposing the microarray to the volatile buffer.

17. The method of claim 11, wherein the volatile buffer is ammonium acetate.

18. The method of claim 17, where in the ammonium acetate has a concentration range of 0.1 molar to 7.5 molar.

19. The method of claim 18, where in the ammonium acetate has a concentration range of 0.5 molar to 2 molar.

20. The method of claim 11, wherein the evaporating of residue volatile buffer is aided by a stream of gas.

21. The method of claim 20, wherein the gas is argon gas.
